(19) United States
(12) Patent Application Publication
(10) Pub. No.: US 2017/0143370 A1
(43) Pub. Date: May 25, 2017

(54) SURGICAL INSTRUMENT UTILIZING ULTRASONIC WAVES

(71) Applicant: Ligasonix, Inc.

(72) Inventor: Eung Kook KIM, Seocho-gu (KR)

(21) Appl. No.: 15/122,397

(22) PCT Filed: Feb. 28, 2014
(86) PCT No.: PCT/KR2014/001706

§ 371 Date: Jan. 30, 2017

(51) Int. Cl. A61B 17/32

(52) U.S. Cl. CPC A61B 17/320092; A61B 2017/320088; A61B 2017/320076

(57) ABSTRACT

The present invention relates to a surgical instrument utilizing ultrasonic waves, the surgical instrument comprising: a transmitting rod having a cylindrical bar shape so as to transmit ultrasonic waves generated in a vibrator connected at one end of the transmitting rod to an amputator extending from the other end; a rod cover covering the transmitting rod with a separating distance from the outer circumferential surface thereof; and a contact prevention unit, provided on the inner circumferential surface of the rod cover, for preventing the transmitting rod from coming into contact with the rod cover due to the vibration generated on the transmitting rod during the transmission of the ultrasonic waves.

### TECHNICAL FIELD

The present invention relates to a surgical instrument utilizing ultrasonic waves, and more particularly, the present invention relates to a surgical instrument that can transmit ultrasonic waves stably while minimizing the transmission loss.

### BACKGROUND ART

Recently, surgical apparatuses using ultrasonic waves are being developed. As one of the representative surgical instruments used in laparoscopic surgery, development of harmonic scalpel is notable.

In a conventional surgical instrument utilizing ultrasonic waves, illustrated in FIG. 1, which can be represented by a harmonic scalpel, ultrasonic waves generated by a vibrator provided inside of a body 4 are transmitted to an amputator 2 through a transmitting rod 1 so that the amputator 2 can cut or stop bleeding of a surgical site.

For your information, in a conventional surgical instrument utilizing ultrasonic waves configured as described above, the motion of jaws 3 gripping a surgical site together with an amputator 2 and the intensity of the ultrasonic waves transmitted to the amputator 2 are controlled by various switches 5 mounted on a body 4. Since the configuration has not much to do with the problems of the conventional surgical instruments utilizing ultrasonic waves, detailed description is omitted.

The problems of the conventional surgical instruments utilizing ultrasonic waves that may occur in the process of transmitting the ultrasonic waves through rod 1 to amputator 2 generated by the vibrator installed inside of the body 4 are described in detail below by referring to FIGS. 2 through 5.

Here, FIG. 2 is a plan view showing the transmission rod and rod cover of an example illustrated in FIG. 1, FIG. 3 is an exploded plan view of a portion of FIG. 2, FIG. 4 is an enlarged perspective view of part A of FIG. 3, and FIG. 5 is an enlarged perspective view of part B of FIG. 3.

As shown in FIGS. 2 through 5, a conventional surgical instrument utilizing ultrasonic waves comprises: a transmission member 1 comprising a transmission rod 10 transmitting ultrasonic waves, a rod cover 20 covering the transmission rod 10, an outer cover 40 covering the rod cover 20, and contact with prevention units 31-33 that fix the position of the transmission rod 10 between the transmission rod 10 and rod cover 20 and prevent the transmission rod 10 and rod cover 20 from contacting with each other by the vibration caused by the transmission of the ultrasonic waves.

Here, the contact prevention units 31˜33 are configured to contact with the outer circumferential surface of the transmission rod 10, and their specific shapes and functions may vary by their positions on the transmission rod 10.

That is, the contact prevention units 31 through 33 can be classified into a first contact prevention unit 31, a second contact prevention unit 32, and a third contact prevention unit 33, which is described in further detail below referring to FIGS. 3 through 5.

Firstly, the first contact prevention unit 31 is provided mainly to fix the transmission rod 10 inside of the rod cover 20 and formed in a circular ring shape as shown in FIG. 5. In order to prevent loss of ultrasonic waves in the process of fixing the transmission rod 10 in the rod cover 20, the first contact prevention unit 31 is located at a portion of the transmission rod 10 corresponding with a plurality of oscillating nodes that are formed in the transmission rod 10 when the rod conveys ultrasonic waves.

Secondly, the second contact prevention unit 32 is provided mainly to prevent the transmission rod 10 from contacting with the rod cover 20 by the vibration generated when the transmission rod 10 conveys ultrasonic waves, and formed in a long, circular pipe shape as shown in FIG. 3. The second contact prevention unit 32 is located at a portion where the probability of the transmission rod 10 to contact with the rod cover 20 is the highest when the transmission rod conveys ultrasonic waves.

Lastly, the third contact prevention unit 33 is provided mainly to enable the transmission rod 10 to fix the rod cover 20 close to the point where the transmission rod 10 and rod cover 20 are bound by each other by the means of coupling hole 12 and coupling pin 14, and formed in an elliptical ring shape as shown in FIG. 4. The third contact prevention unit 33, different from the first contact prevention unit 31, is located close to the coupling hole 12 of the transmission rod 10 with a greater thickness, as the transmission rod 10 and the rod cover 20 contact with each other directly by the coupling pin 14.

Since all the three contact prevention units 31 through 33 described above are attached directly on to the transmission rod 10 that conveys ultrasonic waves in a circular configuration along on the outer circumferential surface of the transmission rod 10, a portion of the ultrasonic waves transmitted through the transmission rod 10 may be absorbed and attenuated by the contact prevention units 31 through 33, even though the contact prevention units are located on the parts where there are less probabilities of ultrasonic waves being absorbed, e.g., oscillating nodes.

That is, the conventional surgical instruments utilizing ultrasonic waves are provided with the contact prevention units 31 through 33 which are attached on the transmission rod 10 at the locations where there are less loss of ultrasonic wave energies when the transmission rod 10 contacts with the rod cover 20 to prevent the transmission rod 10 from contacting with the rod cover 20 at the locations where there are higher loss of ultrasonic wave energies when the transmission rod 10 and the rod cover contact with each other. However, the problem still persists since the configuration cannot minimize the loss of ultrasonic wave energy while the ultrasonic waves are transmitted through the transmission rod 10.

In addition to the problem, the conventional surgical instruments utilizing ultrasonic waves, even though they may be provided with the contact prevention units 31 through 33, there is the possibility that the transmission rod 10 may be damaged because the transmission rod 10 may contact with the rod cover 20 by the vibration of the transmission rod 10 while it conveys ultrasonic waves, which can result in failure to transmission ultrasonic waves stably.

### DETAILED DESCRIPTION OF THE INVENTION

### Technical Objects

To solve above described problems, an aspect of the present invention is to provide a surgical instrument utilizing ultrasonic waves, and more particularly, a surgical instrument that can transmit ultrasonic waves stably while minimizing the transmission loss.

The invention is not restricted to the technical objective set forth above. The above and other aspects of the invention not described herein will become apparent to those skilled in the art to which the invention pertains by referencing the detailed description of the invention below.

### Means for Achieving the Technical Object

A surgical instrument utilizing ultrasonic waves in accordance with the present invention devised to solve above described technical object can comprise: a transmission rod formed in a cylindrical bar shape to convey ultrasonic waves generated by a vibrator connected to one end to an amputator formed by extending from the opposite end; A rod cover for covering the transmission rod, by being isolated from the outer circumferential surface of the transmission rod; and contact prevention units provided on the inner circumferential surface of the rod cover to prevent the transmission rod from contacting with the rod cover by the vibration caused by the transmission of the ultrasonic waves.

Here, the contact prevention units can be provided on the inner circumferential surface of the rod cover at locations opposite to certain points of a plurality of oscillating nodes formed on the transmission rod while the rod conveys the ultrasonic waves,

In addition, the contact prevention units can be provided on the inner circumferential surface of the rod cover at locations opposite to a center point of the transmission rod facing with one half of the transmission rod, with reference to the center point between one end and the opposite end of the rod, including the opposite end.

In addition, while the contact prevention units are provided on the inner circumferential surface of the rod cover corresponding with certain nodal points of a plurality of nodal points formed on the transmission rod when the rod conveys ultrasonic waves, a majority of them can be provided on the inner circumferential surface of the cover rod facing with a portion of the transmission rod corresponding with one half of the rod, with reference to the center point of the one and the opposite ends of the transmission rod, including the opposite end.

Meanwhile, the contact prevention units can be configured to have lengths which are smaller than one fourth of the distance between two adjacent nodal points of oscillation among a plurality of nodal points.

In addition, the length of a contact prevention unit provided on the inner circumferential surface of the rod cover facing with a portion of the transmission rod corresponding with one half of the rod including an opposite end, with reference to the center point between one and an opposite ends of the transmission rod can be configured to be longer than the contact prevention unit provided on the inner circumferential surface of the rod cover facing with another portion of the transmission rod corresponding with one half of the length including one end.

Meanwhile, the contact prevention units may be formed with polytetrafluoroethylene (PTFF) or silicone.

In addition, the contact prevention units may be fixed to or not fixed to the inner circumferential surface of the rod cover.

Meanwhile, the contact prevention units can be provided on the inner circumferential surface of the rod cover contacting with both the transmission rod and rod cover, regardless of the transmission of ultrasonic waves, at the points adjacent to the one and the opposite ends of the transmission rod, wherein the points are of a plurality of the nodal points of oscillation formed on the transmission rod while it is conveying ultrasonic waves.

Here, the contact prevention unit formed at a point adjacent to one end of the transmission rod can be formed on the inner circumferential surface of the rod cover with only a portion of the inner circumferential surface of the rod cover contacting with both the transmission rod and rod cover.

In addition, the contact prevention unit formed at a point adjacent to one end of the transmission rod can be formed in an O-ring shape along the circumference of a groove formed on the rod cover to accommodate a connecting pin that couples the transmission rod and the rod cover.

### Effect of the Invention

The surgical instrument utilizing ultrasonic waves in accordance with an embodiment of the present invention can transmit ultrasonic waves stably minimizing transmission loss, since contact prevention units are provided on the inner circumferential surface of the rod cover surrounding the transmission rod that transmits ultrasonic waves.

The effect of the invention is not restricted to those set forth above. The above and other aspects of the invention will become apparent to those skilled in the art to which the invention pertains by the description of the claims.

### BRIEF DESCRIPTION OF THE DRAWING

FIG. 1 is a perspective view of a conventional surgical instrument utilizing ultrasonic waves,

FIG. 2 is a plan view showing a portion of transmission rod and rod cover of the conventional surgical instrument of FIG. 1,

FIG. 3 is an exploded plan view of a portion of the surgical instrument of FIG. 2,

FIG. 4 is an enlarged view of the part A of FIG. 3,

FIG. 5 is an enlarged perspective view of the part B of FIG. 3,

FIG. 6 is an exploded plan view of a portion of the surgical instrument in accordance with an embodiment of the present invention including the transmission rod and rod cover,

FIG. 7 is a partial perspective, cross-sectional view taken from line I-I of FIG. 6,

FIG. 8 is a cross-sectional, frontal view of FIG. 7,

FIG. 9 is a cross-sectional view of a transformed first contact prevention unit of FIGS. 7 and 8,

FIG. 10 is an enlarged perspective view of a portion of the part C of FIG. 6, and

FIG. 11 is a cross-sectional view taken from line II-II of FIG. 6.

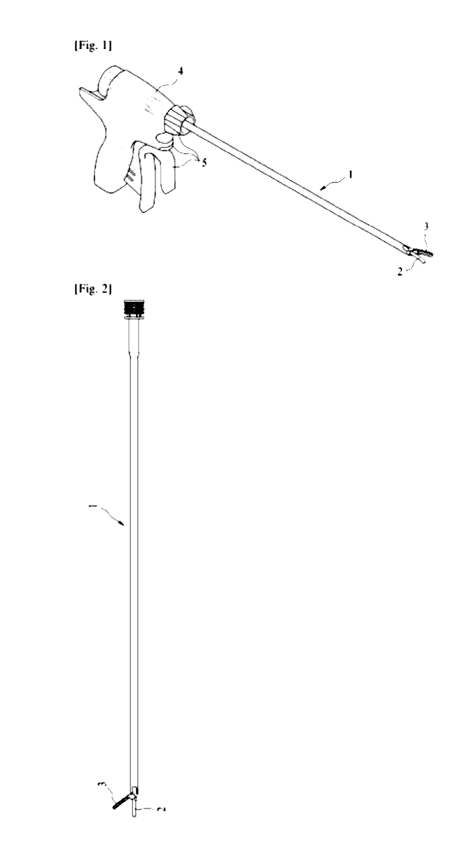

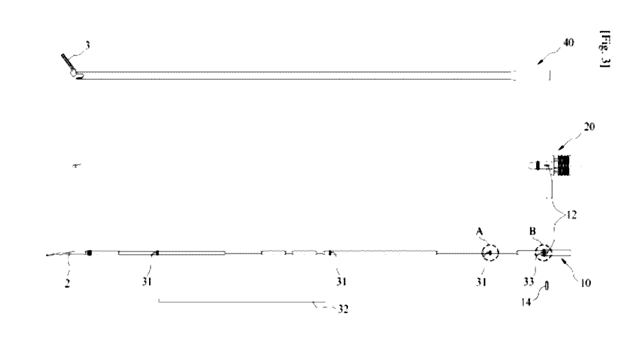

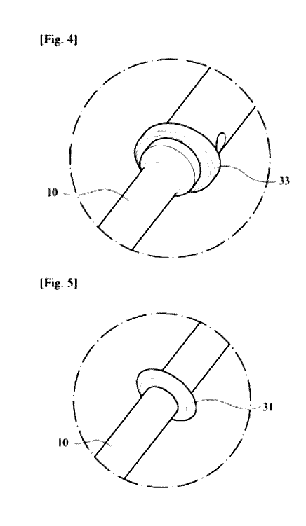

<Numbering Scheme of the Major Parts of the Drawings>

| 100: Transmission rod | |
|---|---|
| 110: One end of transmission rod | |
| 112: Insertion groove | 114: Connecting pin |

120: Opposite end of transmission rod
130: Center of a portion of the transmission
200: Rod cover
300: Contact prevention unit
310: First contact prevention unit
320: Second contact prevention unit
330: Third contact prevention unit
332: Third contact prevention unit formed in an O-ring shape.

### MODE FOR CARRYING OUT THE INVENTION

An embodiment of the present invention is described hereinbelow by referring to the accompanying drawings. In the description of the present invention, known functions and/or configuration can be omitted in order to clarify the spirit of the present invention.

In the description of the present invention, the terms referring to directions, i.e., up, down, or front, rear, are used solely to help those skilled in the art understand the present invention more clearly. As such, such directions are on a relative basis and shall not be interpreted to restrict the scope of the right of the present invention.

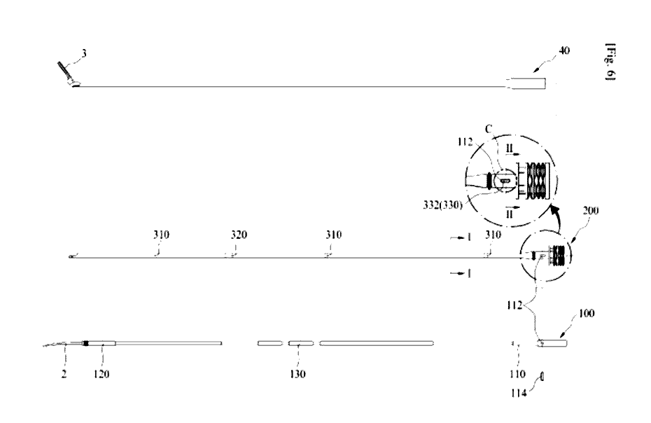

To begin with, the configuration of a surgical instrument in accordance with an embodiment of the present invention is described in detail hereinbelow by referring to FIGS. 6 through 11.

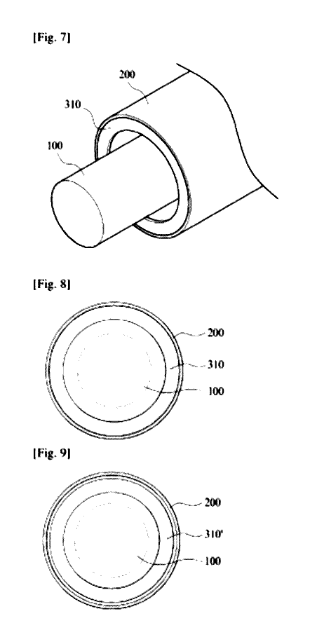

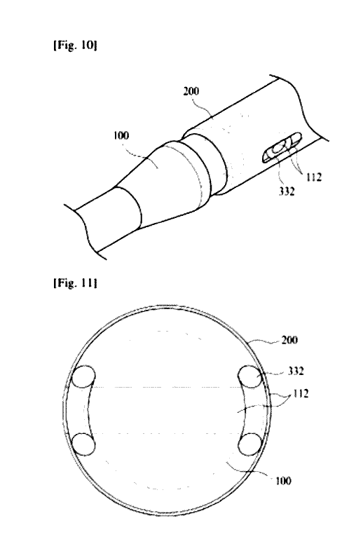

FIG. 6 is an exploded plan view of a portion of the surgical instrument in accordance with an embodiment of the present invention including the transmission rod and rod cover, FIG. 7 is a partial perspective, cross-sectional view taken from line I-I of FIG. 6, FIG. 8 is a cross-sectional, frontal view of FIG. 7, and FIG. 9 is a cross-sectional view of a transformed first contact prevention unit of FIGS. 7 and 8, In addition, FIG. 10 is an enlarged perspective view of a portion of the part C of FIG. 6, and FIG. 11 is a cross-sectional view taken from line II-II of FIG. 6.

Referring to FIGS. 6 through 11, a surgical instrument in accordance with an embodiment of the present invention comprises a transmission rod 100, rod cover 200, and contact prevention units 300.

The transmission rod 100 is a member configured to transmit the ultrasonic waves generated by a vibrator provided on one end 110 to an amputator 2 formed by extending from an opposite end 120 of the transmission rod.

The transmission rod 100 can be configured in a diversified ways without restrictions in its shape, length, material, etc., as long as it can transmit ultrasonic waves from the one end 110 to the opposite end 120.

For example, the transmission rod 100 can be configured in a long, thin, cylindrical bar to minimize transmission loss of ultrasonic waves and can be inserted in a hole formed on a trocar used in laparoscopy to reach a surgical site easily.

In addition, the length of the transmission rd 100 can be provided sufficiently to allow repetition of the vibration nodes (the points where the intensity of ultrasonic wave is substantially zero) and vibration anti-node (a concept opposite to the vibration node, the points where the intensity of ultrasonic wave is the highest) by a plurality of numbers.

In addition, the transmission rod 100 can be formed with a titanium material for effective transmission of the ultrasonic waves generated by a vibrator at a frequency of 55.5 kHz.

Meanwhile, the rod cover 200 is a member surrounding the transmission rod 100 being separated from the outer circumferential surface of the transmission rod 100 to protect the transmission rod 100 from being damaged while the transmission rod 100 transmits ultrasonic waves.

The configuration of the rod cover 200 including shape, thickness, etc., is not limited as far as it can surround the transmission rod 100 being separated from the outer circumferential surface of the transmission rod 100.

That being said, the rod cover 200 would be preferably formed in a thin and long cylindrical shape as the transmission rod 100 is preferable configured in a thin and long shape to be able to be inserted in the hole formed on a trocar to reach a surgical site easily.

The contact prevention units 300 is a member provided to prevent the transmission rod 100 from contacting with the rod cover 200 by the vibration of the transmission rod 100 generated while the transmission rod 100 conveys ultrasonic waves.

The contact prevention units 300, different from the contact prevention units 31 through 33 included in the conventional surgical instruments, wherein the contact prevention units are attached to the transmission rod 100m are attached on the inner circumferential surface of the rod cover 200.

Since the contact prevention units 300 are attached on the inner circumferential surface of the rod cover 200, instead of the outer circumferential surface of the transmission rod 100, different from the conventional surgical instruments utilizing ultrasonic waves, the surgical instrument in accordance with an embodiment of the present invention can minimize the transmission loss of ultrasonic waves in the transmission rod 100.

More particularly, even at the points of vibration nodes where the absorption of ultrasonic waves is small, the intensity of the ultrasonic waves transmitted through the transmission rod 100 can be reduced in a configuration wherein the contact prevention units are contacting with the transmission rod 100. Furthermore, the transmission rod 100 can be damaged. In the surgical instrument in accordance with an embodiment of the present invention, since the contact prevention units 300 is attached on the rod cover 200, not on the transmission rod 100, the surgical instrument is free of the transmission loss of ultrasonic waves and damage of the transmission rod 100.

The contact prevention units 300 can be made of any material and covered by the scope of the right of the present invention as long as they can prevent the transmission rod 100 from contacting directly with the rod cover 200.

For further detailed description, the contact prevention units 300 can be formed with polytetrafluoroethylene (FIFE) or silicone.

PTFE which is a well-known material by its brand name Teflon of Du Pont is a noncombustible fluoric polymer. PTFE is suitable for preparing the contact prevention units 300 due to its low friction coefficient and high resistance to chemicals.

Silicone is a polymer of silicon and oxygen in various formations. Silicone constituted with linear polydimethylsiloxane and oligosiloxane molecules is a representative polymer resin. Silicone is suitable for preparing the contact prevention units 300 due to its low oxidation rate, stability in high temperature, and good sealing property

The contact prevention units 300 may be fixed to or not fixed to the inner circumferential surface of the rod cover 200 as long as they are provided on the inner circumferential surface of the rod cover 200.

More particularly, the contact prevention units 300 can be fixed by being attached onto the inner circumferential surface of the rod cover 200 as shown in FIG. 8, or the contact prevention units 300 can be positioned by being isolated from the inner circumferential surface of the rod cover 200 by a certain gap.

However, if the contact prevention units 300 are isolated from the inner circumferential surface of the rod cover 200 by a certain distance, they are not contacting with the transmission rod 100, which will be described in further detail in relation with the positions and length of the contact prevention units 300.

In addition, the contact prevention units 300 are covered by the patent right of the present invention regardless of their position and/or length, as long as they are provided on the inner circumferential surface of the rod cover 200.

That is, as described hereinbelow, the positions of the contact prevention units 300 do not have to be specified on a specific portion on the inner circumferential surface of the rod cover 200, rather, the contact prevention units 300 can constitute the whole inner circumferential surface of the rod cover 200 without impeding the right of the present invention.

However, for further detailed description hereinbelow, an embodiment of the present invention wherein the contact prevention units 300 comprising the first contact prevention unit 310, second contact prevention unit 320 and third contact prevention unit 330 having diversified lengths are located at various points or regions of the inner circumferential surface of the rod cover 200 will be described.

The first contact prevention unit 310 can be provided on the inner circumferential surface of the rod cover 200 facing with a portion or all of the nodal points of the vibration, where the intensity of the ultrasonic waves is substantially zero, formed on the transmission rod 100 while the transmission rod 100 is transmitting ultrasonic waves.

While this configuration can prevent the transmission rod 100 from contacting with the rod cover 200 using the first contact prevention unit 310, since the transmission rod 100 contacts with the first contact prevention unit 310 in the process, —the transmission rod 100 does not contact with the first contact prevention unit 310 when the transmission rod 100 is not prevented from contacting with the rod cover 200—because the loss of the ultrasonic waves can be minimized by arranging the vibration node of the transmission rod 100 to contact with the first contact prevention unit 310.

As described above, when the first contact prevention unit 310 is provided on the inner circumferential surface of the rod cover 200 facing with the vibration node of the transmission rod 100, the first contact prevention unit 310 can be configured to have a length smaller than ¼ of the distance between two adjacent vibration nodes of a plurality of the vibration nodes.

That is, the length of the first contact prevention unit 310 can be smaller than one half of a half wavelength of the ultrasonic wave transmitted by the transmission rod 100, which is an embodiment configured taking the wavelength of the ultrasonic wave transmitted by the transmission rod 100 into consideration.

The second contact prevention unit 320 can be provided on the inner circumferential surface of the rod cover 200 facing with the center of a portion of the transmission rod 100 which is one half of the length between the center 130 of the one end 110 and the opposite end 120 of the transmission rod 100 and the opposite end 120, that is, at the center of the portion of the transmission rod 100 between the center point 130 of the transmission rod and the amputator 2.

The second contact prevention unit 320 is positioned taking the possibility of contacting between the transmission rod 100 and the rod cover 200 into consideration. This is because, even though the outer circumferential surface of the transmission rod 100 and the inner circumferential surface of the rod cover 200 are configured not to contact with each other, the one end of the transmission rod 100 cannot but contact with the rod cover 200, resulting in a cantilever beam structure of the transmission rod, and the possibility of the transmission rod contacting with the rod cover becomes in the portion farther from the one end 110 and closer to the opposite end 120 due to increased moment caused by the vibration of ultrasonic waves.

That is, the second contact prevention unit 320 is located at a position where the possibility of transmission rod 100 and rod cover 200 contacting with each other is the highest. If it is provided together with the first contact prevention unit 310, ultrasonic waves can be transmitted to the amputator 2 through the transmission rod 100 stably at minimum loss.

Here, since the length of the second contact prevention unit 320 is determined to reduce the probability of the contact between the transmission rod 100 and rod cover 200, the length may preferably be longer than that of the first contact prevention unit 310, different from the method for determining the length of the first contact prevention unit 310 taking the wavelength of the ultrasonic waves into consideration.

That is, the length of the second contact prevention unit 320 can be determined longer than that of another contact prevention unit (e.g., first contact prevention unit 310) provided on the inner circumferential surface of the rod cover 200 facing with another portion of the transmission rod 100 corresponding with one half of the length including the one end 110 of the transmission rod 100.

Meanwhile, similar with the reason applied to the positioning of the second contact prevention unit 320, the first contact prevention unit 310 may be formed by a longer length on the inner circumferential surface of the rod cover 200 facing with a portion of the transmission rod 100 corresponding with one half of the rod including the opposite end 120 than that formed on the inner circumferential surface of the rod cover 200 facing with a portion of the transmission rod 100 corresponding with the one half of the length between the center 130 between the one end 110 and the opposite end 120 and the one end 110 of the transmission rod 100.

As described above, if the transmission rod 100 is not prevented from contacting with the rod cover 200, the contact prevention units 300 and the transmission rod 100 can be so configured as not to contact with each other, however, the present invention is not limited to such configuration.

That is, as the case may be, when the transmission rod 100 is not prevented from contacting with the rod cover 200, the contact prevention units 300 can be provided on the inner circumferential surface of the rod cover 200 and at the same time contacting with the transmission rod 100.

More particularly, the contact prevention units 300 can be provided on the inner circumferential surface of the rod cover 200 contacting with both the transmission rod 100 and the rod cover 200 in the region close to the one end 110 and the opposite end 120 of the transmission rod 100 of the plurality of the vibration nodes on the transmission rod 100.

The purpose of this configuration is to make use of the contact prevention units 300 as a support structure so that the rod cover 200 can surround the transmission rod 100 in a more stable structure.

The third contact prevention unit 330 illustrated in FIGS. 10 and 11 is the representative contact prevention unit 300 wherein the contact prevention unit 300 is contacting with both the transmission rod 100 and rod cover 200.

That is, the third contact prevention unit 330 which is a contact prevention unit 300 provided at a position close to one end 110 of the transmission rod 100 can be provided on the inner circumferential surface of the rod cover 200 having only a portion of the inner circumferential surface of the rod cover 200 is contacting with both the transmission rod 100 and rod cover 200.

More particularly, the third contact prevention unit 330 can be formed in an O-ring shape along an insertion groove 112 formed on the rod cover 200 to be inserted with a coupling pin 114 which couples the transmission rod 100 and rod cover 200.

This configuration wherein the third contact prevention unit 330 allows only a portion of the inner circumferential surface of the rod cover 200 contacting with both the transmission rod 100 and rod cover 200—contacting only around the insertion groove 112 formed on the rod cover 200—intends to minimize the contacting area between the contact prevention unit 300 and transmission rod 100 even though the contact prevention unit 300 plays a role of support structure.

As described above, the position, length and shape of the contact prevention units 300 can be diversified, however, even though the contact prevention units 300 are configured in diversified ways, all such configurations do not depart from the scope of the present invention as long as the contact prevention units 300 are provided on the inner circumferential surface of the rod cover 200 to prevent direct contact between the transmission rod 100 and rod cover 200.

Because the surgical instruments in accordance with the embodiments of the present invention utilizing ultrasonic waves comprises contact prevention units 300 different from the conventional surgical instruments utilizing ultrasonic waves, they can transmit ultrasonic waves stably at minimized loss.

While an embodiment of the present invention is described by referring to a harmonic scalpel, it would be obvious that the present invention can be applied to other types of surgical instruments utilizing ultrasonic waves, and further, other medical systems such as computer-integrated robotic surgery systems.

From the foregoing it is believed that those skilled in the pertinent art will recognize the meritorious advancement of this invention and will readily understand that while the present invention has been described in association with a preferred embodiment thereof, and in the accompanying drawings, numerous changes and modifications may be made therein by those skilled in the art without departing from the spirit and scope of this invention. Therefore, the modifications and changes shall not be interpreted independently from the technical spirit and point of view, and the modified embodiments shall be interpreted to be within the scope of the present invention.

What is claimed is:

1. A surgical instrument utilizing ultrasonic waves, comprising:
a transmission rod formed in a cylindrical bar to transmit ultrasonic waves generated by a vibrator coupled to one end to an amputator formed by extending from an opposite end;
a rod cover for covering the transmission rod, by being isolated from the outer circumferential surface of the transmission rod; and
contact prevention units provided on the inner circumferential surface of the rod cover to prevent the transmission rod from contacting with the rod cover by the vibration caused by the transmission of the ultrasonic waves.

2. A surgical instrument utilizing ultrasonic waves of claim 1,
wherein the contact prevention units are substantially provided on the inner circumferential surface of the rod cover at locations facing with certain points of a plurality of vibration nodes formed on the transmission rod while the rod conveys the ultrasonic waves.

3. A surgical instrument utilizing ultrasonic waves of claim 1,
wherein the contact prevention units can be provided on the inner circumferential surface of the rod cover at locations facing with a center point of the transmission rod corresponding with one half of the transmission rod, with reference to the center point between the one end and the opposite end of the rod, including the opposite end.

4. A surgical instrument utilizing ultrasonic waves of claim 1,
wherein, while the contact prevention units are provided on the inner circumferential surface of the rod cover corresponding with certain nodal points of a plurality of nodal points formed on the transmission rod when the rod conveys ultrasonic waves, a majority of them can be provided on the inner circumferential surface of the cover rod facing with a portion of the transmission rod corresponding with one half of the rod, with reference to the center point of the one and the opposite ends of the transmission rod, including the opposite end.

5. A surgical instrument utilizing ultrasonic waves of claim 1,
wherein the contact prevention units are configured to have lengths which are smaller than one fourth of the distance between two adjacent vibration nodes among a plurality of the vibration nodes.

6. A surgical instrument utilizing ultrasonic waves of claim 1,
wherein the length of a contact prevention unit provided on the inner circumferential surface of the rod cover facing with a portion of the transmission rod corresponding with one half of the rod including an opposite end, with reference to the center point between one and an opposite ends of the transmission rod is configured to be longer than the contact prevention unit provided on the inner circumferential surface of the rod cover facing with another portion of the transmission rod corresponding with one half of the length including one end.

7. A surgical instrument utilizing ultrasonic waves of claim 1,
wherein the contact prevention units are formed with polytetrafluoroethylene (PTFE).

8. A surgical instrument utilizing ultrasonic waves of claim 1,
wherein the contact prevention units are formed with silicone.

9. A surgical instrument utilizing ultrasonic waves of claim 1,
wherein the contact prevention units are fixed on the outer circumferential surface of the rod cover.

10. A surgical instrument utilizing ultrasonic waves of claim 1,
wherein the contact prevention units are provided on the inner circumferential surface of the rod cover contacting with both the transmission rod and rod cover, regardless of the transmission of ultrasonic waves, at the points adjacent to the one and the opposite ends of the transmission rod, wherein the points are of a plurality of the vibration nodes formed on the transmission rod while it is conveying ultrasonic waves.

11. A surgical instrument utilizing ultrasonic waves of claim 10,
wherein the contact prevention unit formed at a point adjacent to one end of the transmission rod is formed on the inner circumferential surface of the rod cover with only a portion of the inner circumferential surface of the rod cover contacting with both the transmission rod and rod cover.

12. A surgical instrument utilizing ultrasonic waves of claim 11,
wherein the contact prevention unit formed at a point adjacent to one end of the transmission rod is formed in an O-ring shape along the circumference of a groove formed on the rod cover to accommodate a connecting pin that couples the transmission rod and the rod cover.
